(19) United States
(12) Patent Application Publication
(10) Pub. No.: US 2022/0108802 A1
(43) Pub. Date: Apr. 7, 2022

(54) TECHNIQUES FOR SELECTING AND PRESENTING A CUSTOMIZED HEALTH RELATED FEATURE FOR A USER OF AN INPUT/OUTPUT DEVICE

(71) Applicant: Intuition Robotics, Ltd, Ramat-Gan (IL)

(72) Inventors: Shay ZWEIG, Harel (IL); Alex KEAGEL, Tel Aviv (IL); Itai MENDELSOHN, Tel Aviv (IL); Roy AMIR, Mikhmoret (IL); Dor SKULER, Oranit (IL); Yoav CASPIN, Tel Aviv (IL)

(73) Assignee: Intuition Robotics, Ltd., Ramat-Gan (IL)

(21) Appl. No.: 17/495,195

(22) Filed: Oct. 6, 2021

Related U.S. Application Data
(60) Provisional application No. 63/088,072, filed on Oct. 6, 2020.

(51) Int. Cl. G16H 50/20

(52) U.S. Cl. CPC G16H 50/20; G16H 20/00

(57) ABSTRACT

A method for identifying and presenting a customized health-related feature for a user of a digital assistant. The method includes: determining, based on an input dataset, a current state of a user and a desirability score; generating a first group of a plurality of health-related features based on the current state, wherein the plurality of health-related features within the first group shares at least a common characteristic among the plurality of health-related features; extracting an experience level value for each of the plurality of health-related features within the first group; identifying a customized health-related feature; and presenting the customized health-related feature by the digital assistant.

### CROSS-REFERENCE TO RELATED APPLICATIONS

This application claims the benefit of U.S. Provisional Application No. 63/088,072, filed on Oct. 6, 2020, the contents of which are hereby incorporated by reference.

### TECHNICAL FIELD

The disclosure generally relates to digital assistants operated in an input/output (I/O) device, and more specifically to a technique for customizing and presenting health-related feature.

### BACKGROUND

As manufacturers improve the functionality of devices such as vehicles, computers, mobile phones, appliances, and the like, through the addition of digital features, manufacturers and end-users may desire enhanced device functionalities. The manufacturers, as well as the relevant end-users, may desire digital features which improve user experiences, interactions, and features which provide for greater connectivity. Certain manufacturers may include device-specific features, such as setup wizards and virtual assistants, to improve device utility and functionality. Further, certain software packages may be added to devices, either at the point of manufacture, or by a user after purchase, to improve device functionality. Such software packages may provide functionalities including, as examples, a computer system's voice control, facial recognition, biometric authentication, and the like.

While the features and functionalities described hereinabove provide for certain enhancements to a user's experience when interacting with a device, the same features and functionalities, as may be added to a device by a user or manufacturer, fail to include certain aspects which may allow for a further-enhanced user experience. First, certain currently-implemented digital assistants and other user experience features may fail to provide for adaptive adjustment of the operation of the assistant or feature. For example, a digital assistant configured to play music may be programmed to use a specific type of music streaming services, thereby limiting the user experience. In addition, certain currently-implemented digital assistants and other user experience features may fail to provide for adjustment of assistant or feature operation based on context or environmental data. As an example, a digital assistant may be configured to present reminders to take vitamins at a certain time. However, such reminder may be inappropriate and disturbing when the user is surrounded by guests or at an important meeting.

Moreover, currently-implemented digital assistants and other user experience features may fail to provide for adaptive adjustment of assistant based on each user and their changes. Often times the policies or services presented by such digital assistance are selected based on fast search, general availability, or at best, based on past information about a single user. As such, users may not benefit from policies or actions performed or suggested by the digital assistants, and eventually abandon the usage of such device.

It would therefore be advantageous to provide a solution that would overcome the challenges noted above.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for identifying a customized health-related feature by a digital assistant. The method comprises: determining, based on an input dataset, a current state of a user and a desirability score; generating a first group of a plurality of health-related features based on the current state, wherein the plurality of health-related features within the first group shares at least a common characteristic among the plurality of health-related features; extracting an experience level value for each of the plurality of health-related features within the first group; identifying a customized health-related feature; and presenting the customized health-related feature by the digital assistant.

Certain embodiments disclosed herein also include a non-transitory computer readable medium having stored thereon causing a processing circuitry to execute a process, the process comprising: determining, based on an input dataset, a current state of a user and a desirability score; generating a first group of a plurality of health-related features based on the current state, wherein the plurality of health-related features within the first group shares at least a common characteristic among the plurality of health-related features; extracting an experience level value for each of the plurality of health-related features within the first group; identifying a customized health-related feature; and presenting the customized health-related feature by the digital assistant.

Certain embodiments disclosed herein also include a system for identifying a customized health-related feature by a digital assistant. The system comprises: a processing circuitry; and a memory, the memory containing instructions that, when executed by the processing circuitry, configure the system to: determine, based on an input dataset, a current state of a user and a desirability score; generate a first group of a plurality of health-related features based on the current state, wherein the plurality of health-related features within the first group shares at least a common characteristic among the plurality of health-related features; extract an experience level value for each of the plurality of health-related features within the first group; identify a customized health-related feature; and present the customized health-related feature by the digital assistant.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a network diagram utilized to describe the various embodiments of the disclosure.

FIG. 2 is a block diagram of a controller, according to an embodiment.

FIG. 3 is a flowchart illustrating a method for presenting a customized health-related feature for a user according to an embodiment.

FIG. 4 is a flowchart illustrating a method for identifying a customized health-related feature according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

The various disclosed embodiments present techniques for effectively and accurately identifying and presenting a customized health-related feature for a user of a digital assistant. The customized health-related feature is determined based on input data including at least a historical data and real-time data of a specific user, as well as a real-time data of the user's environment. Based on these input data, a health-related feature that optimally suits the user's need at a specified moment in real-time and/or near real-time may be presented by the digital assistant. The disclosed embodiments allow objective selection of the customized health-related feature based on experience level values that increase accuracy and consistency.

The embodiments disclosed herein provide specific improvement in adaptive adjustment to user and user environment. By collecting and analyzing real-time data and historical data of a user and their environment, the current state of the user is accurately and effectively determined. In this case, a current desirability score can be determined to efficiently assess whether to advance into analyses, and thus preserve and reduce processing power at the digital assistant.

Moreover, in an embodiment, multiple health-related features may be clustered according to various characteristics and states of the user to objectively identify the customized health-related feature based on factors such as experience level value and priority scores. It should be noted that such factors can be continuously and/or periodically updated using, for example, monitoring and user feedback, to further customize and adaptively adjust to the user. While the user may manually update and input preferences and information, such manual process is time consuming, inconsistent, and is, ultimately, a subjective process. The embodiment disclosed herein provide for objective identification of customized health-related feature application for a specific user at a specific time and thus significantly reduce false output.

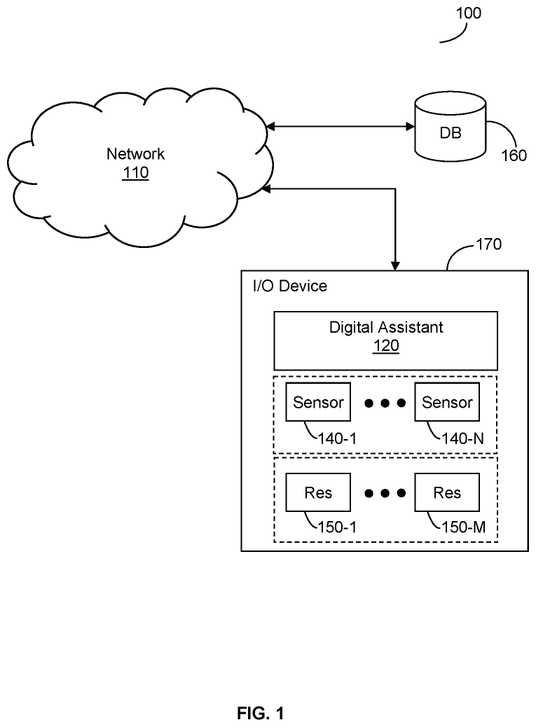

FIG. 1 is an example network diagram 100 utilized to describe the various disclosed embodiments. The network diagram 100 includes an input/output (I/O) device 170 operating a digital assistant 120. In some embodiments, the digital assistant 120 is further connected to a network 110 to allow some processing of a remote server (e.g., a cloud server). The network 110 may provide for communication between the elements shown in the network diagram 100. The network 110 may be, but is not limited to, a local area network (LAN), a wide area network (WAN), a metro area network (MAN), the Internet, a wireless, cellular, or wired network, and the like, and any combination thereof.

In an embodiment, the digital assistant 120 may be connected to, or implemented on, the I/O device 170. The I/O device 170 may be, for example and without limitation, a robot, a social robot, a service robot, a smart TV, a smartphone, a wearable device, a vehicle, a computer, a smart appliance, and the like.

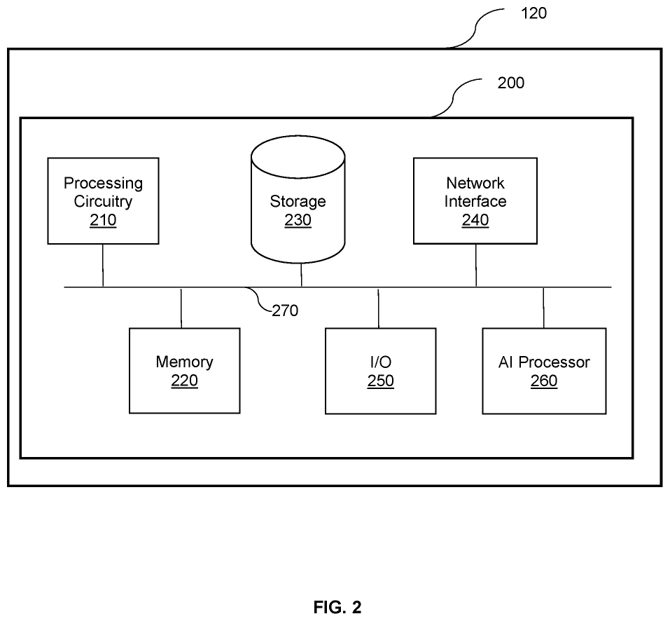

The digital assistant 120 may be realized in software, firmware, hardware, and any combination thereof. An example block diagram of a controller that may execute the processes of the digital assistant 120 is provided in FIG. 2. The digital assistant 120 is configured to process sensor data collected by one or more sensors, 140-1 to 140-N, where N is an integer equal to or greater than 1 (hereinafter referred to as “sensor” 140 or “sensors” 140 for simplicity) and one or more resources 150-1 to 150-M, where M is an integer equal to or greater than 1 (hereinafter referred to as “resource” 150 or “resources” 150 for simplicity). The resources 150 may include, for example, electro-mechanical elements, display units, speakers, and the like. In an embodiment, the resources 150 may include sensors 140 as well. The sensors 140 and the resources 150 are included in the I/O device 170.

The sensors 140 may include input devices, such as various sensors, detectors, microphones, touch sensors, movement detectors, cameras, and the like. Any of the sensors 140 may be, but are not necessarily, communicatively, or otherwise connected to the digital assistant 120 (such connection is not illustrated in FIG. 1 for the sake of simplicity and without limitation on the disclosed embodiments). The sensors 140 may be configured to sense signals received from a user interacting with the I/O device 170 or the digital assistant 120, signals received from the environment surrounding the user, and the like. In an embodiment, the sensors 140 may be implemented as virtual sensors that receive inputs from online services, for example, the weather forecast, a user's calendar, and the like.

In an embodiment, the network diagram 100 further includes a database (DB) 160. The database 160 may be stored within the I/O device 170 (e.g., within a storage device not shown), or may be separate from the I/O device 170 and connected thereto via the network 110. The database 160 may be utilized for storing, for example, historical data about one or more users, users' preferences and related policies, and the like, as well as any combination thereof.

According to various disclosed embodiments, the digital assistant 120 is configured to determine a customized health-related feature for a user, at that specified moment and output at the I/O device 170, as further discussed below. To this end, health-related features are clustered into sub-datasets that include health-related features associated with certain characteristics of current state. Based on experience level values and priority scores for each health-related feature, the customized health-related feature may be objectively and accurately identified by the digital assistant 120 to result in an optimal health-related feature for the specific user at the immediate moment. Moreover, a current desirability score is determined and compared against a predetermined threshold to ensure appropriate timing for presenting the customized health-related feature to the user of the I/O device 170. In this scenario, the customization is performed not only on user preference, but user environment based on historical data and real-time data for effective and accurate output as described in more detail below.

FIG. 2 is an example block diagram of a controller 200 acting as a hardware layer of a digital assistant 120, according to an embodiment. The controller 200 includes a processing circuitry 210 that is configured to receive data, analyze data, generate outputs, and the like, as further described hereinbelow. The processing circuitry 210 may be realized as one or more hardware logic components and circuits. For example, and without limitation, illustrative types of hardware logic components that can be used include field programmable gate arrays (FPGAs), application-specific integrated circuits (ASICs), application-specific standard products (ASSPs), system-on-a-chip systems (SOCs), general-purpose microprocessors, microcontrollers, digital signal processors (DSPs), and the like, or any other hardware logic components that can perform calculations or other manipulations of information.

The controller 200 further includes a memory 220. The memory 220 may contain therein instructions that, when executed by the processing circuitry 210, can cause the controller 200 to execute actions as further described hereinbelow. The memory 220 may further store therein information, e.g., data associated with one or more users, historical data about one or more users, users' preferences and related policies, and the like.

The storage 230 may be magnetic storage, optical storage, and the like, and may be realized, for example, as a flash memory or other memory technology, or any other medium which can be used to store the desired information.

In an embodiment, the controller 200 includes a network interface 240 that is configured to connect to a network, e.g., the network 110 of FIG. 1. The network interface 240 may include, but is not limited to, a wired interface (e.g., an Ethernet port) or a wireless port (e.g., an 802.11 compliant Wi-Fi card), configured to connect to a network (not shown).

The controller 200 further includes an input/output (I/O) interface 250 configured to control the resources (150, FIG. 1) which are connected to the digital assistant 120. In an embodiment, the I/O interface 250 is configured to receive one or more signals captured by the sensors (140, FIG. 1) of the digital assistant 120 and to send such signals to the processing circuitry 210 for analysis. In an embodiment, the I/O interface 250 is configured to analyze the signals captured by the sensors 140, detectors, and the like. In a further embodiment, the I/O interface 250 is configured to send one or more commands to one or more of the resources 150 for presenting customized health-related features of the digital assistant 120, as further discussed herein below. A health-related feature may be for example, an on-line service such as a service that enables users to meet a doctor on-line instead of going to the doctor's clinic, a downloadable application allowing to improve the human memory, a downloadable application allowing to practice yoga on-line, and so on. In further embodiment, the components of the controller 200 are connected via a bus 270.

In some configurations, the controller 200 may further include an artificial intelligence (AI) processor 260. The AI processor 260 may be realized as one or more hardware logic components and circuits, including graphics processing units (GPUs), tensor processing units (TPUs), neural processing units, vision processing units (VPU), reconfigurable field-programmable gate arrays (FPGA), and the like. The AI processor 260 is configured to perform, for example, machine learning based on sensory inputs received from the I/O interface 250, where the I/O interface 250 receives input data, such as sensory inputs, from the sensors 140.

According to various disclosed embodiments, the controller 200 is configured to collect a dataset about at least the user of the digital assistant 120. The dataset may include real-time data, as well as historical data about the user and the user's environment. The real-time data may be sensed and collected using one or more sensors (e.g., sensors 140, FIG. 1), and may include, for example, images, videos, audio signals, and the like, that are captured in real-time or near real-time with respect to the user. The real-time data may indicate the user's mood, the specific location of the user, whether the user is awake or asleep, and the like. The historical data may include, for example, user's behavioral patterns, user's routines, user's preferences, and so on. In a non-limiting example, the historical data may indicate that the user takes a certain medication on a daily basis and suffers from overweight. In further embodiment, the controller 200, when executing the digital assistant 120, may be configured to collect real-time data about the user's environment, such as the current number of people near the user, the time, the current weather, the temperature outside the user's home or vehicle, traffic condition, and so on. In an embodiment, the datasets, or a portion of it, may be inputted by the user using a user interface.

According to a further embodiment, the set of data may be collected from one or more electronic sources such as, but not limited to, a database (e.g., database 160, FIG. 1), social media websites, the user's electronic calendar, and so on. It should be noted that the dataset may be collected constantly or periodically.

In an embodiment, the controller 200, when executing the digital assistant 120, is configured to apply the collected dataset to at least one algorithm, such as a machine learning algorithm to determine a current state of the user interacting with the I/O device 170 and a current desirability score. According to further embodiment, the dataset may be analyzed using, for example and without limitations, one or more computer vision techniques, audio signal processing techniques, machine learning techniques, and the like.

The current state may reflect the state of the user and the state of the environment near the user in real-time, or near real-time. The current state may indicate whether, for example, the user is sleeping, reading, stressed, angry, or other actions or emotional behaviors. The current state may further indicate the current time, weather, number of people in the room, people's identity, and so on. As an example, the current state may indicate that the user is sitting in the living room, that three other people are sitting next to the user, the identity of the other three people, and that the time is 7:30 pm.

The desirability score that is associated with the current state of the user may be generated to determine the level of appropriateness to select and present a customized health-related feature to the user at the respective time. A desirability score greater than the predetermined desirability score objectively decides that the time is appropriate for presenting and thus, advances to the analyses for identifying a customized health-related feature. As noted above, the health-related feature may be for example, an on-line service such as a service that enables users to meet a doctor on-line instead of going to the doctor's clinic, a downloadable application allowing to improve the human memory, a downloadable application allowing to practice yoga on-line, and so on. It should be noted that the desirability score comparison is a decision point for the analyses to prevent data overload and overworking of the controller 200.

In a non-limiting example, the determined current state may indicate that the user is sleeping, the time is 4 am, and the desirability score is less than the predetermined threshold, and thus, it may not be desirable to present any feature as it may be very disturbing for the user. However, when the user is awake, the time is 11 am, the desirability score is greater than the predetermined threshold, and the user asks the digital assistant (e.g., digital assistant 120, FIG. 1) to call to the doctor in order to coordinate an appointment, it may be desirable to suggest an applicable feature (or service) such as meeting the doctor on-line.

In an embodiment, the controller 200, when executing the digital assistant 120, is configured to identify a customized health-related feature at that immediate time for the specific user of the I/O device 170 from a plurality of health-related features. The plurality of health-related features may be stored in a database (e.g., the database 160) including a list of predetermined features such as on-line services, downloadable applications, new features of existing applications, and so on. It should be noted that the customized health-related feature is determined to be most appropriate for the user at the specific time (or moment) as further discussed herein.

In an embodiment, selecting the customized health-related feature may be achieved based on analysis of the collected datasets (including real-time data and historical data of the user and the user's environment), an experience level value with respect to each of the plurality of health-related features, and a health-related features' priority list, as further discussed herein below. As noted above, for example, the dataset may indicate, among other things, medications the user usually takes, the general physical and mental health of the user, real-time mental health, real-time physical health, user's preferences, user's behavioral patterns, user's habits, and so on.

In an embodiment, the plurality of health-related features may be clustered into small groups (or sub-datasets) at the controller 200 based on the characterization of each of the health-related features. That is, for example, health-related features that are online yoga applications may be clustered into the same group. In another example, applications or on-line services that are relating to making a doctor's appointment such as on-line appointment service, user calendar, and more, may be in the same group. More specifically, a first group of health-related features may be generated based on the aggregated datasets from the user that include real-time and historical data of the user. In further embodiment, the determined current state of the user may be used as a basis for generating the first group of health-related features.

The experience level value may indicate the user's knowledge level (and/or usage level) with respect to each of the plurality of health-related features in the determined first group. The experience level value may be a number from “1” to “5” where “1” is the lowest value indicating that, for example, the user is not familiar with the feature at all (or that the user did not use it for a long time), “2” indicating that the user used the feature only once, “3” means that the user used the feature in several occasions recently, “4” means that the user uses the feature frequently and “5” means that the user uses the feature on a daily basis. In an embodiment, historical and current experience level values that are associated with each of the health-related features may be constantly collected and calculated such that an accurate determination of the experience level value is achieved.

As an example, a user may frequently use a first health-related feature and after a few months the user stops using the first feature. According to the same example, although the experience level value was “4” for a few months, after the user stopped using the feature for several weeks the experience level value may be updated to be “3” indicating the lack of usage. According to the same example, in case the user did not use the feature for more than three months, the user experience level value may be updated once again and this time the experience level value may be “1”. The different experience level values may indicate whether it is desirable to present the feature for the first time (e.g., for “1” experience level values), whether it is desirable to encourage the user to use the feature more frequently (e.g., for “3” experience level values), and so on.

In an embodiment, a health-related feature (e.g., a suggested on-line service) may be rejected by the user. Upon determination that the user rejected a particular feature, the controller 200, when executing the digital assistant 120, may be configured to stop suggesting the particular feature or alternatively using a different persuasive technique for suggesting the user to use the feature. According to a further embodiment, the experience level value of each of the plurality of predetermined health-related features may be determined by monitoring the set of data about at least the user. Monitoring the set of data about the user may occur over a predetermined time period.

According to another embodiment, the controller 200, when executing the digital assistant 120, may be configured to determine a current status of each health-related feature. A status of a health-related feature may indicate, for example, whether the health-related feature has been already discovered by the user, undiscovered, rejected by the user, adopted, or abandoned. As an example, after the user uses a specific health-related feature for more than ten times a month for more than three months, the status of the specific feature may be “adopted”. In a further embodiment, the controller 200, when executing the digital assistant 120, may be configured to use the abovementioned experience level values as an input in order to determine the current status of each health-related feature.

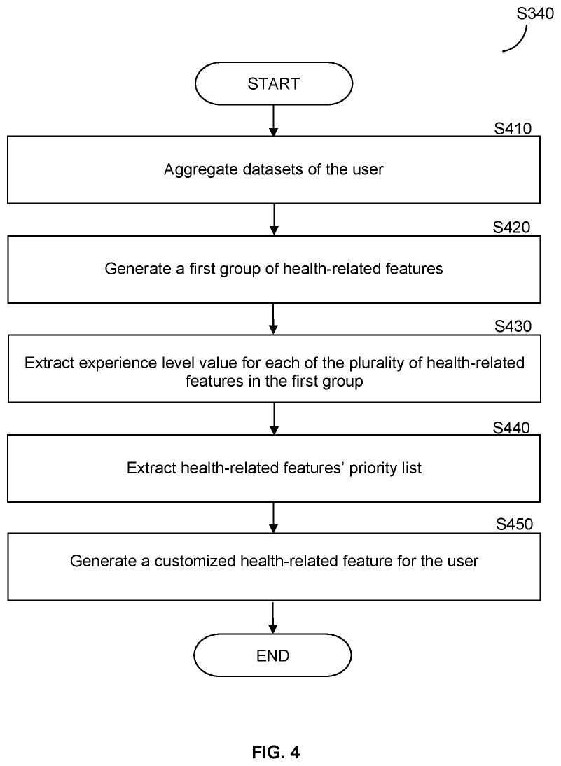

The health-related features' priority list may include a ranking of the plurality of features indicating which of the health-related features should be promoted more intensively compared to the rest of the plurality of features. The health-related features' priority list may be previously determined and periodically updated with a priority score that is associated with each of the health-related features. In an example, a first on-line yoga application may be promoted more intensively due to a higher priority score compared to a second on-line yoga application having a lower priority score. According to another example, a first medical on-line service may be located higher at the health-related features' priority list and therefore promoted more intensively compared to a second medical on-line service that is located in a lower position. It should be noted that the health-related features' priority list may be previously determined and periodically updated with a priority score that is associated with each of the health-related features based on, for example, one or more recommendations received from a medical care team. That is, based on the recommendations of the medical team (or any other authorized entity) a first health-related feature may be promoted more intensively compared to a second health-related feature. The process of selecting a customized health-related feature to be presented to the user is discussed in further detail herein below with respect to FIG. 4.

In an embodiment, the controller 200, when executing the digital assistant 120, presented the identified customized health-related feature. Presenting the customized health-related feature may be performed using one or more resources (e.g., the resources 150) that are communicatively connected to and controlled by the digital assistant 120. Such resources may be for example, a speaker, a display unit, a smartphone that is communicatively connected to the electronic social agent, and so on.

As an example, a dataset is collected and analyzed and therefore indicating that the user has just started to suffer from a back pain, the time is 4 pm, and the user is alone at home. According to the same example, since the current state of the user indicates that the user is awake, alone at home, and in pain, the controller 200, when executing the digital assistant 120, determines that it is desirable to select and present a customized health-related feature to the user. According to the same example, the controller 200, when executing the digital assistant 120, is configured to extract historical data of the user, user experience level value with respect to each of the plurality of predetermined health-related features, and a health-related features' priority list. According to the same example, the historical data may indicate that the user usually goes to the doctor's clinic every three months. The user experience level value with respect to an on-line service that enables to meet the doctor on-line is low, which means that the user is probably not aware of this option.

The health-related features' priority list may include more than one option related to the similar on-line service however, one of the similar on-line services (e.g., that enables meeting a doctor on-line) may have a higher priority score and therefore may be selected instead of the other similar on-line services. According to the same example, the controller 200 aggregates the extracted data and analyzes the aggregated data in order to select and present the most appropriate feature for the user, such as, a particular on-line service enabling the user to meet a doctor on-line.

In another embodiment, the controller 200 may be configured to collect and analyze at least one user feedback (e.g., user response) with respect to the presented customized health-related feature. The feedback may be collected using one or more sensors (e.g., the sensors 140). The feedback may indicate whether the user accepts the presented customized health-related feature, whether the user rejects the presented health-related feature, what is the level of rejection, and so on. In one embodiment, the analysis may be achieved using, for example and without limitations, one or more computer vision techniques, audio signal processing techniques, machine learning techniques, and the like.

In a further embodiment, the controller 200, when executing the digital assistant 120, may be configured to perform at least one action based on at least one of the abovementioned experience level value of each of the plurality of predetermined health-related features and corresponding user feedback. An action may be, for example, generating a reminder for using at least one of the plurality of health-related features, setting a health-related feature as a default, preventing the presentation of a health-related feature, and so on. As an example, when the user feedback indicates that the user reacted in a very negative manner to a suggestion to use a new health-related feature, an action that prevents the presentation of the particular health-related feature may be performed in order to stop bothering the user in future cases. On the contrary, in case the user feedback indicates that the user politely rejected the suggestion (e.g., the presented health-related feature), the action may include generating a reminder to use the health-related feature in the future. As another example, when it is determined with respect to a particular health-related feature that the user experience level value is the highest possible (e.g., “5” out of “1” when “5” is the highest), indicating that the user uses the feature a lot, the controller 200 may take an action that includes setting the particular health-related feature as a default health-related feature for the user.

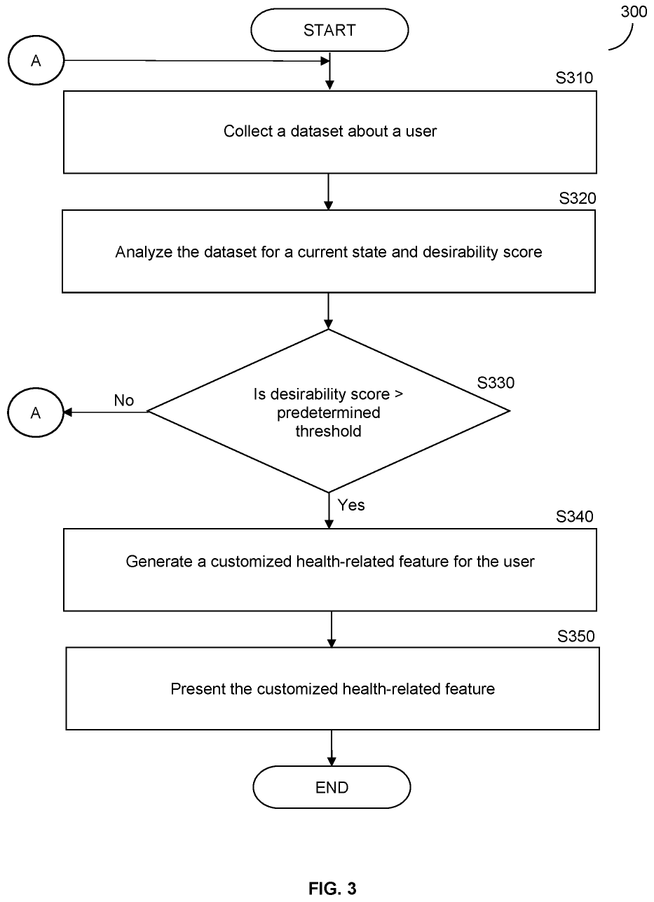

FIG. 3 shows an example flowchart 300 illustrating a method for presenting a customized health-related feature at an I/O device according to an embodiment. The method described herein may be executed by the controller 200 that is further described herein above with respect to FIG. 2.

At S310, datasets associated with a user of the I/O device is collected. The datasets may include real-time data as well as historical data about the user and the user's environment. The dataset may include for example, images, videos, audio signals, and the like, that are captured with respect to the user. The real-time data may indicate, for example, the user's mood, physical condition, location, and the like and the historical data may include user's information such as preference, routines, behavioral patterns, and the like. Furthermore, real-time data about the user's environment may present, for example, the temperature of the home (inside and/or outside), number of people in the room, and more. In an embodiment, the real-time data may include sensory data collected by the various sensors (e.g., 140, FIG. 1) of the I/O device.

At S320, the collected dataset is analyzed to determine a current state of the user and a current desirability score. The analysis may be achieved using at least one algorithm, such as a machine learning algorithm. A machine learning algorithm may be trained based on historical data related to users. The current state may reflect the condition of the user and the condition of the environment in a predetermined proximity to the user in real-time, or near real-time. The current state may indicate whether, for example, the user is injured, not feeling good, crying, stressed, angry, sleeping, and so on. The current state may further indicate the current time, the weather, the number of people in the room, and the like. In addition to the current state of the user, the desirability score is determined objectively to identify the appropriate timing for selecting and presenting a customized health-related feature to the user. The desirability score may indicate the degree of appropriateness with respect to factors such as, but not limited to, current time, people around the user, weights of condition parameters, combinations thereof, and the like. In other configurations, the collected dataset may be analyzed using, for example and without limitations, one or more computer vision techniques, audio signal processing techniques, unsupervised machine learning techniques, and the like.

At S330, a check is performed whether the desirability score is greater than a predetermined threshold. If so, execution continues with S340; otherwise, execution continues with S310. In an example embodiment, the predetermined threshold score may be stored at the memory of the controller 200.

At S340, the customized health-related feature is identified based on analysis of dataset obtained regarding the user. The customized health-related feature may be selected from a plurality of health-related features. Selecting the customized health-related feature may be achieved based on analysis of the collected datasets of the user, an experience level value with respect to each of the plurality of health-related features, and a health-related features' priority list. The process of identifying the customized health-related feature is discussed in greater detail with respect to FIG. 4 below. The customized health-related feature, in an embodiment, is the recommended health-related feature that is applicable for the user at real-time or near real-time.

At S350, the identified customized health-related feature is presented to the user. Presenting the customized health-related feature may be performed using one or more resources (e.g., the resources 150) that are communicatively connected to and controlled by the controller of the digital assistant (120, FIG. 1)

FIG. 4 is an example flowchart S340 illustrating a method for identifying a customized health-related feature according to an embodiment.

At S410, datasets associated with a user of the I/O device is aggregated. The datasets may include, for example, the historical data, the real-time data as well as plurality of health-related features. The dataset may include information about the user for example, user's patterns, habits, chronic diseases, and so on. The real-time data may be collected from one or more sensors, from a database (e.g., the database 160), websites, social media, user's calendar, and so on. Each health-related feature may be, for example, an on-line service such as a service that enables users to meet a doctor on-line instead of going to the doctor's clinic, a downloadable application allowing to improve the human memory, a downloadable application allowing to practice yoga on-line, and so on.

At S420, a first group of health-related features is generated. The plurality of health-related features may be clustered into smaller groups (or sub-datasets) with respect to characteristics, for example, method of interaction, type of service, function, and so on. To this end, the first group may include one or more health-related features that correspond to the current need of the user as determined based on the aggregated datasets of the user. In an embodiment, the first group may include multiple health-related features that correspond to the current state of the user. In an example, the time is 8 am, the user is awake, and is expressing back pain. In this scenario, the first group may include health-related features, such as, but not limited to, multiple online yoga practice applications, multiple Pilates practice applications, and an online service to make an appointment for a doctor and/or chiropractor. It should be noted that a certain health-related feature may be clustered into more than one group (or sub-dataset).

At S430, an experience level value for each of the plurality of health-related features within the first group is extracted. The experience level value may be extracted from a database (e.g., the database 160). The experience level value may indicate, with respect to each health-related feature, whether the user is familiar with the feature, uses the feature a lot, not familiar with the feature at all, used the feature only once, etc., as further discussed herein above with respect to FIG. 2.

It should be appreciated that the experience level value is a numerical value indicating familiarity and/or frequency of user that enables objective comparison of the plurality of health-related features. In another embodiment, a current status of each health-related feature may be determined based on the experience level values of each health-related feature. The current status of each health-related feature may indicate, for example, whether the health-related feature has been discovered already by the user, undiscovered, rejected by the user, adopted, or abandoned.

At S440, a priority list for the plurality health-related features within the first group is extracted. The health-related features' priority list may be extracted from, for example, a database (e.g., the database 160). The priority list may include a ranking of each of the plurality of health-related features within the respective first group indicating which of the features should be promoted more intensively compared to the rest of the plurality of features. The health-related features' priority list may be previously determined and periodically updated with a priority score that is associated with each of the health-related features.

According to one embodiment, the health-related features' priority list may be previously determined and periodically updated with a priority score that is associated with each of the health-related features based on, for example, one or more recommendations received from a medical care team. That is, based on the recommendations of the medical team (or any other authorized entity) a first health-related feature may be promoted more intensively compared to a second health-related feature. According to another embodiment, the priority list may be adjusted based on, for example, an external population data indicating a health-related feature more likely to be suitable for the user in a certain age group or sex, for example. That is, a population to which the user is associated with may be detected and the most common health-related features of the population may be given higher ranking in the priority list. It should be noted that the population to which the user may be associated may be detected based on analysis of the abovementioned datasets of the user, which may include population data that may be extracted from one or more data sources.

At S450, a customized health-related feature is generated for the user. In an embodiment, the historical data, the real-time data, as well as extracted experience level data, and the priority list may be input into a machine learning model that is trained to determine a customized health-related feature for the user. In an embodiment, a fit score may be determined for each of the plurality of health-related features within the first group based on factors such as experience level values, weights depending on ranking in priority list, combinations thereof, and the like. In such an embodiment, a health-related feature with the highest fit score may be identified as the customized health-related feature that is appropriate for the user of the digital assistant at the specific time (or moment).

The various disclosed embodiments are implemented as hardware, firmware, software, or any combination thereof. Moreover, the software is preferably implemented as an application program tangibly embodied on a program storage unit or computer readable medium. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosure and the concepts contributed by the inventor to furthering the art and are to be construed as being without limitation to such specifically recited examples and conditions. Moreover, all statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for identifying a customized health-related feature by a digital assistant, comprising:
determining, based on an input dataset, a current state of a user and a desirability score;
generating a first group of a plurality of health-related features based on the current state, wherein the plurality of health-related features within the first group shares at least a common characteristic among the plurality of health-related features;
extracting an experience level value for each of the plurality of health-related features within the first group;
identifying a customized health-related feature; and
presenting the customized health-related feature by the digital assistant.

2. The method of claim 1, wherein the customized health-related feature is selected based on the current state and the experience level value for each of the plurality of health-related features.

3. The method of claim 1, wherein identifying the customized health-related feature further comprises:
extracting a priority list of the plurality of health-related features within the first group, wherein the priority list ranks the plurality health-related features including priority scores for each of the plurality of health-related features; and
determining a fit score based on the experience level value and the priority list for each of the plurality of health-related features.

4. The method of claim 1, wherein the experience level value is a number generated by continuous monitoring of the input dataset over a predetermined time period.

5. The method of claim 1, further comprising:
collecting at least one user feedback with respect to the identified customized health-related feature; and
analyzing the at least one user feedback.

6. The method of claim 5, further comprising:
performing at least one action based on experience level value and the collected at least one user feedback.

7. The method of claim 5, wherein the at least one action comprises at least one of:
generating a reminder for using at least one of the plurality of health-related features, setting the at least one of the plurality of health-related feature as a default, and preventing the presentation of the at least one of the plurality of health-related feature.

8. The method of claim 1, further comprising:
determining the desirability score based on the input dataset, wherein the customized health-related feature is not presented when the determined desirability score is smaller than a predetermined threshold.

9. The method of claim 1, wherein the input dataset includes at least one of: a real-time data and a historical data.

10. A non-transitory computer readable medium having stored thereon instructions for causing a processing circuitry to execute a process, the processing comprising:
determining, based on an input dataset, a current state of a user and desirability score;
generating a first group of a plurality of health-related features based on the current state, wherein the plurality of health-related features within the first group shares at least a common characteristic among the plurality of health-related features;
extracting an experience level value for each of the plurality of health-related features within the first group;
identifying a customized health-related feature; and
presenting the customized health-related feature by the digital assistant.

11. A system for identifying a customized health-related feature by a digital assistant, comprising:
a processing circuitry; and
a memory, the memory containing instructions that, when executed by the processing circuitry, configure the system to:
determine, based on an input dataset, a current state of a user and a desirability score;
generate a first group of a plurality of health-related features based on the current state, wherein the plurality of health-related features within the first group shares at least a common characteristic among the plurality of health-related features;
extract an experience level value for each of the plurality of health-related features within the first group;
identify a customized health-related feature; and
present the customized health-related feature by the digital assistant.

12. The system of claim 11, wherein the customized health-related feature is selected based on the current state and the experience level value for each of the plurality of health-related features.

13. The system of claim 11, wherein identifying the customized health-related feature further comprises:
extracting a priority list of the plurality of health-related features within the first group, wherein the priority list ranks the plurality health-related features including priority scores for each of the plurality of health-related features; and
determining a fit score based on the experience level value and the priority list for each of the plurality of health-related features.

14. The system of claim 11, wherein the experience level value is a number generated by continuous monitoring of the input dataset over a predetermined time period.

15. The system of claim 11, wherein the system is further configured to:
collect at least one user feedback with respect to the identified customized health-related feature; and
analyze the at least one user feedback.

16. The system of claim 15, wherein the system is further configured to:
perform at least one action based on experience level value and the collected at least one user feedback.

17. The system of claim 15, wherein the at least one action comprises at least one of: generating a reminder for using at least one of the plurality of health-related features, setting the at least one of the plurality of health-related feature as a default, and preventing the presentation of the at least one of the plurality of health-related feature.

18. The system of claim 11, wherein the system is further configured to:
determine the desirability score based on the input dataset, wherein the customized health-related feature is not presented when the determined desirability score is smaller than a predetermined threshold.

19. The system of claim 11, wherein the input dataset includes at least one of: a real-time data and a historical data.
